(19) United States
(12) Patent Application Publication
(10) Pub. No.: US 2016/0289412 A1
(43) Pub. Date: Oct. 6, 2016

(54) DEGRADABLE ISOCYANATE COMPOUNDS AND APPLICATIONS THEREOF

(71) Applicant: ADESSO ADVANCED MATERIALS WUXI CO., LTD., Wuxi,, Jiangsu (CN)

(72) Inventors: Bing Qin, Shanghai (CN); Xin Li, Cambridge (GB); Bo Liang, Plainsboro, NJ (US)

(73) Assignee: ADESSO ADVANCED MATERIALS WUXI CO., LTD., Wuxi,, Jiangsu (CN)

(21) Appl. No.: 15/038,705

(22) PCT Filed: Dec. 2, 2014
(86) PCT No.: PCT/CN2014/001082

§ 371 Date: May 23, 2016

(30) Foreign Application Priority Data
Dec. 2, 2013 (CN) 201310634385.2

(51) Int. Cl. C08J 11/16; C07C 263/10; C07C 265/04; C07D 317/28; C08G 18/48; C08J 11/28; C08G 18/58; C08J 11/24; C08L 75/08; C08J 5/24; C08K 7/06; C08K 3/04; C07C 265/12; C08G 18/77

(52) U.S. Cl. CPC C08J 11/16; C07C 265/12; C07C 263/10; C07C 265/04; C07D 317/28; C08G 18/4841; C08G 18/771; C08G 18/58; C08J 11/24; C08L 75/08; C08J 5/24; C08K 7/06; C08K 3/04; C08J 11/28; C08J 2375/08; C08L 2201/06; C08L 2203/16; C08L 2205/025; C08L 2207/20; C08G 2230/00

(57) ABSTRACT

Degradable isocyanate compounds, methods for making these compounds, and uses of these compounds for preparing degradable cross-linked polymers and composite materials are provided.

### CROSS-REFERENCE TO RELATED APPLICATION

This application claims priority to Chinese Application Number 201310634385.2, filed on Dec. 2, 2013, the contents of which are incorporated herein by reference in their entireties.

### BACKGROUND OF THE INVENTION

Due to the unique structure and excellent property, polyurethane material of the polyfoam, elastomer and adhesive are widely used in the construction, automobile industry, national defense and aerospace etc.

The backbone of polyurethane contains the high molecular compound with a repeating segment of polyurethane. The polyurethane belongs to the synthetic material which has its wide-range in application. Up to now, the polyurethane with its widely use in the biomedical polymer material is obtained through the process that the prepolymer of the isocyanate terminated matrix, synthesized by the macromolecule dihydric alcohol, and the excess of diisocyanate can combine with low molecular dibasic alcohol or diamine to carry the chain reaction. Thereinto, the macromolecule polyhydric alcohols becomes the flexible chain; diisocyanate and chain-extender turn into the rigid chain. Take the frequently-used diisocyanate and dihydric alcohol as the examples and the reaction formula is as follows:

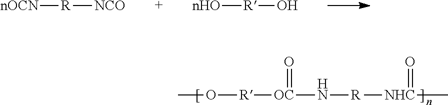

The flexible chain of polyurethane is generally constituted by the polyether or polyester with weak polarity, which reflects its elastic properties. The glass transition temperature, tensile strength, elongation, abrasion resistance, shear strength, blood compatibility and the hydrophilic properties of polyurethane can be regulated by the molecular design and choose different types of flexible chain or different molecular weights of rigid chain, or combine several kinds of flexible chain and rigid chain into the application to make the polyurethane posess the specific properties. The polymerization could generate the block or cross-linked polymer. More than the carbamate included in the polyurethane macromolecule, the ether, ester, urea, biuret, allophanate matrix etc could be also contained. The structures of polyurethane macromolecule are changeful, whose properties could be adjusted over a wide range. Different numbers and different types of functional groups take different synthesis crafts to prepare the polyurethane products with different varieties and properties.

Under natural conditions, the vast majority of plastics and other macromolecule materials currently used are non-degradable and the heavy use of macromolecule materials could cause the serious problems of white pollution, and then the environment was destroyed enormously. With the gradually deteriorating environmental problems of global warming, pollution arised in the earth, the environmental protection is urgently required to take actions to deal with. Now, many countries have made the legislation to restrict the use of non-degradable single-used plastic bags and promote the use of disposable shopping bags, garbage bags made by biodegradable plastic. In the packaging or other industry, polyurethane foam plastics, which possess excellent high specific strength, good insulation properties, fine vibration cushioning properties and other characteristics, can be used as the high-grade packaging materials. Polyurethane material with good biocompatibility anti-thrombotic property has the advantages of excellent mechanical properties, easy processing, low price, etc. So it has a broad application prospects in the biomedical field. But these hardly degradable polyurethane plastics have brought the environmental pollution problems for the industrial development. Therefore, the degradation property of polythurethane material has a crucial importance in the application of packaging and medical industry. For the single-used packaging material, the degradable polyurethane foaming plastics could reduce the environmental pollution to protect the environment. Moreover, the degradable medical polyurethane could be biodegraded and absorbed by human body. And in the meantime, it can be used as the excellent tissue engineering, alternative materials for drug delivery to promote the technical progress of medicine greatly.

Currently, the polyurethane recycling methods contain the physical recycling, incineration recycling and chemical recycling. The physical recycling methods do not destroy the chemical structure of the eupolymer and have not changed its composition. The polyurethane could be reused as the filler, compression molding and other purposes. The physical recycling method of polyurethane is simple to execute. However, the market of the products obtained by this method is limited and the technical limitations of process also arised. The recycling waste is mainly the low-grade scrap recycling polyurethane waste. And the incineration recycling method mainly takes the incineration method to obtain energy from the polyurethane waste, whilst a large amount of toxic would be discharged to cause serious environmental pollution on the condition that the incomplete combustion of polyurethane happens in the incineration process. The purpose of chemical recycling method is to degrade the polyurethane into the reused the liquid oligomer or the organic compound with small molecule under the condition of the chemical reagents and catalysts applied in the polyurethane. The recycling of raw material would be achieved by above-mentioned chemical method. However, due to its limitations of price and cost, the technical skills of degradable polyurethane are still immature with the low marketization and commercialization. So the fundamental research of the degradable polythuerthane technology should be carried out systematically and thoroughly.

Furthermore, compared with the traditional epoxy resin, the polyurethane modified epoxy resin has excellent properties in its toughness and shock strength. Because of the fine mechanical properties, electrical insulation, bonding properties, the polyurethane is widely used in the composite, casting parts, electronic appliances, paint and other industry. Of the fiber-reinforced epoxy composites, the carbon fiber composite has been put into the application of aviation, automobiles, trains, ships, wind power, tidal energy, sporting goods and other industries. By 2015, the global production of composite materials will be increase greatly of over 10 million tons. However, the high density and three-dimensional network structure of cured epoxy resin would make itself become the extremely hard and durable materials, which can withstand the effects of a wide range of environmental conditions. Meanwhile, the cross-linked network structure of cured epoxy resin could hardly make itself removed, recycled to reuse. Essentially, the cross-linked reaction through the process of polyamine and epoxy resin compound is irreversible. Thus this substance can't be remelted, reformed without damage and easily dissolved. And the dealing and recycling of fiber composite waste would be the worldwide problems to constrain the sustainable development of fiber composites.

Up to now, the recovery process of fiber composite has the followed methods: 1. High temperature thermal degradation (Thermochimica Acta 2007(454):109-115), by which clean filler and fibre can be recycled, but needs to be done under the condition of high temperature, requiring high standard of equipment; 2. fluidized bed (Applied surface science 2008 (254): 2588-2593), also needs to be done under the condition of high temperature to recycle the clean fibre; 3. supercutical fluid (water (Materials and design 2010 (31): 999-1002), alcohol (Ind. eng. chem. res. 2010 (49): 4535-4541), carbon dioxide (CN102181071), etc) also achieves the degradation of epoxy resin system, but still in labortray stage and has a long way from the real industrialization; 4. Applying nitric acid (Journal of applied polymer science, 2004 (95): 1912-1916) to degrade the epoxy resin, the recycled fibre is clean, however, strong acid like nitric acid is highly corrosive, requires high standard of equipment, and operation security is low but the recycling cost is high, the post-processing is also difficult to realize. In general, these methods have limitations of different levels, such as fiber shortening, performance degradation, environmental pollution and high cost of recycling. Thus, the effective degradation method for recycling the composite waste is also the urgent problem to be solved in the composite industry.

All references referred to herein are incorporated by reference in their entireties.

### SUMMARY OF THE INVENTION

Aiming at the problems of the existing technology, this applicant provides a method for degradable isocyanate and its preparation ways of the polyurethane modified epoxy resin synthesized by the isocyanate and epoxy resin, the polyurethane and reinforced composites synthesized by the isocyanate and dyhydroxy or polyol compound, the polyurethane modified polymer, the polyurethane polymer and reinforced composite. The degradable composites prepared by using this invention have good mechanical property, and are widely used in the application industry of different composites. Under the specific conditions, the composite could be degraded. And the degradable products of reinforced material and polymer matrix could be also separated and recycled. Moreover, the degradation recovery method of composite could be easily and economically controlled under the mild reaction conditions.

In one aspect, the present invention provides isocyanate compounds having at least two isocyanate groups, wherein each of the isocyanate groups is bonded via a linker to a linear or cyclic oxy-carbohydro moiety that contains oxygen atoms not less than the isocyanate groups and optionally contains one or more heteroatoms each independently being S or N; each of the linker is alkylene, heteroalkylene, alkenylene, heteroalkyl, alkynylene, heteroalkynlene, arylene, or heteroaryl; the linear oxy-carbohydro moiety is alkyl, alkenyl, or alkynyl optionally substituted in the main chain with one or more heteroatoms, and is connected via oxo to each of the linkers that link the isocyanate groups to the oxy-carbohydro moiety; and the cyclic oxy-carbohydro is saturated, unsaturated, or aromatic ring or fused ring containing at least one oxygen atom in the ring and optionally contain an oxygen in a ring substituent that is bonded to the linker with an isocyanate group. As used herein, the term oxy-carbohydro refers to a functional group which has a carbohydro core or moiety that has two or more oxygen atoms each connecting the carbohydro moiety with the linker to an isocyanate group. For illustration, an example of such oxy-carbohydro is CH₃CH(—O—)₂ which contains two oxo group and —CH(CH₃)— as the carbohydro moiety.

In some embodiments, the present invention provides isocyanate compounds of Formula (I) shown below:

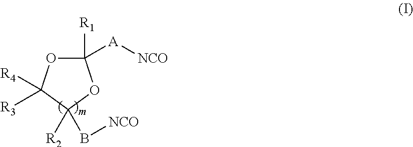

### In Formula (I),

m is 1, 2, 3, 4, or 5;

each of R₁, R₂, R₃ and R₄ independently is hydrogen, alkyl, cycloalkyl, heterocyclic, heterocyclic, alkenyl, cycloalkenyl, alkynyl, aryl, heteroaryl, alkylene-oxy-alkyl, alkylene-oxy-alkyl, alkylene-oxy-hetero-cyclic, alkylene-oxy-hetero-cycloalkyl, alkylene-oxy-alkenyl, alkylene-oxy-cycloalkenyl, alkylene-aryl, alkylene-oxy-heteroaryl, cycloalkylene-oxy-alkyl, cycloalkylene-oxy-cycloalkyl, cycloalkylene-oxy-heterocyclic, cycloalkylene-oxy-heterocycloalkyl, cycloalkylene-oxy-alkenyl, cycloalkylene-oxy-cycloalkenyl, cycloalkylene-oxy-aryl, cycloalkylene-oxy-heteroaryl, heterocycloalkylene-oxy-alkyl, heterocycloalkylene-oxy-cycloalkyl, heterocycloalkylene-oxy-heterocyclic, heterocycloalkylene-oxy-heterocycloalkyl, heterocycloalkylene-oxy-alkenyl, heterocycloalkylene-oxy-cycloalkenyl, heterocycloalkylene-oxy-aryl, heterocycloalkylene-oxy-heteroaryl, arylene-oxy-alkyl, arylene-oxy-cycloalkyl, arylene-oxy-heterocyclic, arylene-oxy-heterocycloalkyl, arylene-oxy-alkenyl, arylene-oxy-cycloalkenyl, arylene-oxy-aryl, or arylene-oxy-heteroaryl; or

R₃ and R₄, together with the carbon atom to which they are bonded, form a 3-7 membered ring optionally containing one or more heteroatoms each of which is independently S, O, or N; or

R₁ and A; together with the carbon atom to which they are bonded, form a 3-7 membered ring optionally containing one or more heteroatoms each of which is independently S, O, or N; or

R₂ and B, together with the carbon atom to which they are bonded, form a 3-7 membered ring optionally containing one or more heteroatoms each of which is independently S, O, or N; or

each of A and B independently is alkylene, alkylene-hetero-alkylene, alkenylene, alkenylene-hetero-alkenylene, alkylene-hetero-alkenylene, alkynylene, cycloalkylene, alkylene-cycloalkylene, alkylene-cycloalkylene-alkylene, alkenylene-cycloalkylene, alkenylene-cycloalkylene-alkenylene, alkylene-cycloalkylene-alkenylene, alkynylene-cycloalkylene, alkynylene-cycloalkylene-alkynylene, heterocycloalkylene, alkylene-heterocycloalkylene, alkylene-heterocycloalkylene-alkylene, alkenylene-heterocycloalkylene, alkenylene-heterocycloalkylene-alkenylene, alkylene-heterocycloalkylene-alkenylene, alkynylene-heterocycloalkylene, alkynylene-heterocycloalkylene-alkynylene, cycloalkenylene, alkylene-cycloalkenylene, alkylene-cycloalkenylene-alkylene, alkenylene-cycloalkenylene, alkenylene-cycloalkenylene-alkenylene, alkylene-cycloalkenylene-alkenylene, alkynylene-cycloalkenylene, alkynylene-cycloalkenylene-alkynylene, heterocycloalkenylene, alkylene-heterocycloalkenylene, alkylene-heterocycloalkenylene-alkylene, alkenylene-heterocycloalkenylene, alkenylene-heterocycloalkenylene-alkenylene, alkylene-heterocycloalkenylene-alkenylene, alkynylene-heterocycloalkenylene, alkynylene-heterocycloalkenylene-alkynylene, Arylene, alkylene-arylene, alkylene-arylene-alkylene, alkenylene-arylene, alkenylene-arylene-alkenylene, alkylene-arylene-alkenylene, alkynylene-arylene, alkynylene-arylene-alkynylene, Heteroarylene, alkylene-heteroarylene, alkylene-heteroarylene-alkylene, alkenylene-heteroarylene, alkenylene-heteroarylene-alkenylene, alkylene-heteroarylene-alkenylene, alkynylene-heteroarylene, alkynylene-heteroarylene-alkynylene, carbonyl, or thiocarbonyl.

In some narrower embodiments, m is 1; each of R₁, R₂, R₃ and R₄ is independently hydrogen or alkyl; or each A and B independently is alkylene or alkenylene.

In some embodiments, the present invention provides isocyanate compounds of Formula (II):

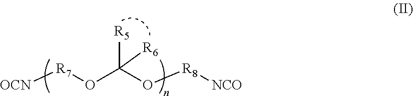

### In Formula (II),

each of R₅ and R₆ independently is hydrogen, alkyl, cycloalkyl, heterocyclic, heterocycloalkyl, alkenyl, cycloalkenyl, aryl, heteroaryl, alkyl-hetero-alkyl, alkynyl, alkylene, alkylene-hetero-alkylene, alkenylene, alkylene-hetero-alkenylene, alkynylene, or alkylene-hetero-alkynylene; or, R₅ and R₆, together with the carbon atom to which they are bonded, form a 3-7 membered ring optionally containing one or more heteroatoms each of which is independently S, O, or N;

n is 1, 2, 3, 4, 5, or 6;

each of R₇ and R₈ is independently alkylene, alkylene-hetero-alkylene, alkenylene, alkenylene-hetero-alkenylene, alkylene-hetero-alkenylene, alkynylene, cycloalkylene, alkylene-cycloalkylene, alkylene-cycloalkylene-alkylene, alkenylene-cycloalkylene, alkenylene-cycloalkylene-alkenylene, alkylene-cycloalkylene-alkenylene, alkynylene-cycloalkylene, alkynylene-cycloalkylene-alkynylene, heterocycloalkylene, alkylene-heterocycloalkylene, alkylene-heterocycloalkylene-alkylene, alkenylene-heterocycloalkylene, alkenylene-heterocycloalkylene-alkenylene, alkylene-heterocycloalkylene-alkenylene, alkynylene-heterocycloalkylene, alkynylene-heterocycloalkylene-alkynylene, cycloalkenylene, alkylene-cycloalkenylene, alkylene-cycloalkenylene-alkylene, alkenylene-cycloalkenylene, alkenylene-cycloalkenylene-alkenylene, alkylene-cycloalkenylene-alkenylene, alkynylene-cycloalkenylene, alkynylene-cycloalkenylene-alkynylene, heterocycloalkenylene, alkylene-heterocycloalkenylene, alkylene-heterocycloalkenylene-alkylene, alkenylene-heterocycloalkenylene, alkenylene-heterocycloalkenylene-alkenylene, alkylene-heterocycloalkenylene-alkenylene, alkynylene-heterocycloalkenylene, alkynylene-heterocycloalkenylene-alkynylene, Arylene, alkylene-arylene, alkylene-arylene-alkylene, alkenylene-arylene, alkenylene-arylene-alkenylene, alkylene-arylene-alkenylene, alkynylene-arylene, alkynylene-arylene-alkynylene, Heteroarylene, alkylene-heteroarylene, alkylene-heteroarylene-alkylene, alkenylene-heteroarylene, alkenylene-heteroarylene-alkenylene, alkylene-heteroarylene-alkenylene, alkynylene-heteroarylene, alkynylene-heteroarylene-alkynylene, 1,4-alkyl substituted piperazine, carbonyl, or thiocarbonyl.

In some other embodiments, each of R₅ and R₆ independently is hydrogen or alkyl; or, R₅ and R₆, together with the carbon atom to which they are bonded, form a 3-6 membered ring optionally containing one or more heteroatoms each of which is independently S, O, or N; n is 1; or each of R₇ and R₈ is independently alkylene, alkylene-hetero-alkylene, or alkenylene.

In still some other embodiments, the present invention provides an isocyante compound of Formula (II):

(R₉)ₚR(—O—R₁₀—N═C═O)_{q} (III)

### In Formula (III),

R and the oxygen atoms (each between R and R₁₀) together constitute the linear or cyclic oxy-carbohydro moiety that optionally contains one or more heteroatoms each independently being S or N;

p is an integer no less than 0;

q is an integer of at least 3; when R is a carbon atom, the sum of p and q is 4;

each R₉ independently is hydrogen, alkyl, cycloalkyl, heterocyclic, heterocycloalkyl, alkenyl, cycloalkenyl, aryl, heteroaryl, alkyl-hetero-alkyl, alkynyl, alkylene, alkylene-hetero-alkylene, alkenylene, alkylene-hetero-alkenylene, alkynylene, or alkylene-hetero-alkynylene; or, two R₉, together with the carbon atom to which they are bonded, form a 3-7 membered ring optionally containing one or more heteroatoms each of which is independently S, O, or N; and

each R₁₀ independently is alkylene, alkylene-hetero-alkylene, alkenylene, alkenylene-hetero-alkenylene, alkylene-hetero-alkenylene, alkynylene, cycloalkylene, alkylene-cycloalkylene, alkylene-cycloalkylene-alkylene, alkenylene-cycloalkylene, alkenylene-cycloalkylene-alkenylene, alkylene-cycloalkylene-alkenylene, alkynylene-cycloalkylene, alkynylene-cycloalkylene-alkynylene, heterocycloalkylene, alkylene-heterocycloalkylene, alkylene-heterocycloalkylene-alkylene, alkenylene-heterocycloalkylene, alkenylene-heterocycloalkylene-alkenylene, alkylene-heterocycloalkylene-alkenylene, alkynylene-heterocycloalkylene, alkynylene-heterocycloalkylene-alkynylene, cycloalkenylene, alkylene-cycloalkenylene, alkylene-cycloalkenylene-alkylene, alkenylene-cycloalkenylene, alkenylene-cycloalkenylene-alkenylene, alkylene-cycloalkenylene-alkenylene, alkynylene-cycloalkenylene, alkynylene-cycloalkenylene-alkynylene, heterocycloalkenylene, alkylene-heterocycloalkenylene, alkylene-heterocycloalkenylene-alkylene, alkenylene-heterocycloalkenylene, alkenylene-heterocycloalkenylene-alkenylene, alkylene-heterocycloalkenylene-alkenylene, alkynylene-heterocycloalkenylene, alkynylene-heterocycloalkenylene-alkynylene, Arylene, alkylene-arylene, alkylene-arylene-alkylene, alkenylene-arylene, alkenylene-arylene-alkenylene, alkylene-arylene-alkenylene, alkynylene-arylene, alkynylene-arylene-alkynylene, Heteroarylene, alkylene-heteroarylene, alkylene-heteroarylene-alkylene, alkenylene-heteroarylene, alkenylene-heteroarylene-alkenylene, alkylene-heteroarylene-alkenylene, alkynylene-heteroarylene, alkynylene-heteroarylene-alkynylene, 1,4-alkyl substituted piperazine, carbonyl, or thiocarbonyl.

Examples of the isocyanate compounds provided by the present invention include bis(4-isocyanatophenoxy)methane, bis(2-isocyanatoethoxy)methane, 2,4-bis(isocyanatomethyl)-1,3-dioxolane, 1,1,1-tris(2-isocyanatoethoxy)ethane, 1,1,2-tris(2-isocyanatoethoxy)ethane, tetrakis(2-isocyanatoethoxy)methane, 1,1,1,2-tetrakis(2-isocyanatoethoxy)ethane, and 4,4′,438-(ethane-1,1,1-triyltris(oxy))tris(isocyanatobenzene).

In still another aspect, the present invention provides a method for preparing an isocyanate compound as described above. The method includes at least the step of converting a compound of Formula (I-A), Formula (II-A), or Formula (III-A) to the isocynate compound, wherein R, R₁, R₂, R₃, R₄, A, B, R₅, R₆, R₇, R₈, R₉, R₁₀, m, n, p, and q, when present, are the same as those ste forth above for the isocyanate compounds of this invention.

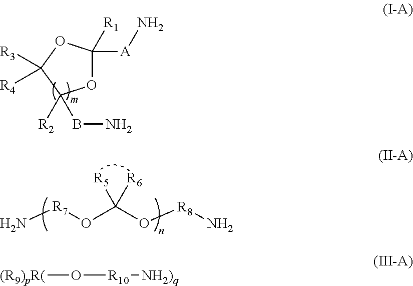

In some embodiments of this method, the conversion of the compound of Formula (I-A), Formula (II-A), or Formula (III-A) to the desired isocyanate compound is by reacting the compound of Formula (I-A), Formula (II-A), or Formula (III-A) with phosgene, triphosgene, or trichloronethyl chloroformate, optionally at the presence of a catalyst. In this reaction, e.g., the molar ratio of the compound of Formula (I-A), Formula (II-A), or Formula (III-A) to phosgene, triphosgene, or trichloronethyl chloroformate is 1:2˜100, the reaction temperature is in the range of −20˜150° C.; and the catalyst, when present, comprises an amine, a pyridine derivative, or N,N dimethyl formamide.

In yet still another aspect, the present invention provides another method for preparing an isocyanate compound described above. The method includes the step of converting a compound of Formula (I-B), Formula (II-B), or Formula (III-B) to the isocyanate compound, wherein R, R₁, R₂, R₃, R₄, A, B, R₅, R₆, R₇, R₈, R₉, R₁₀, m, n, p, and q, when present, are the same as those in the isocyanate compound, and each X is independently hydroxyl, thiol, or trimethylsiloxy.

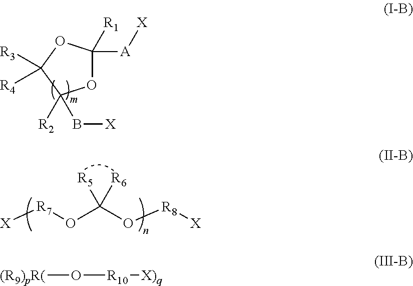

In this method, e.g., the conversion of the compound of Formula (I-B), Formula (II-B) or Formula (III-B) to the desired isocyanate compound can be by reacting the compound of Formula (I-B), Formula (II-B), or Formula (III-B) with tetrabutylammonium cyanate, optionally with the presence of a catalyst. In such a reaction, the molar ratio of the compound of Formula (I-B), Formula (II-B) or Formula (III-B) to tetrabutylammonium cyanate can be in the range of 1:2˜100, the reaction temperature can be in the range of −20˜150° C.; and the catalyst, when present, includes a triazine compound.

In still another aspect, the present invention provides degradable polyurethanes, wherein each of the polyurethanes is made by polymerizing an isocyanate compound as described above with a hydrogen-donating compound which comprises a dihydric alcohol, a polyhydric alcohol, polyetherpolyol, polyesterpolyol, binary mercaptan, polybasic mercaptan, phenol, carboxylic acid, urea, amide, diamine, or polyamine; and the polyurethane has a cleavable cross-linking structure of Formula (I-C), Formula (II-C), or Formula (III-C), wherein R, R₁, R₂, R₃, R₄, A, B, R₅, R₆, R₇, R₈, R₉, R₁₀, m, n, p, and q, when present, are the same as those in the isocyanate compounds described above.

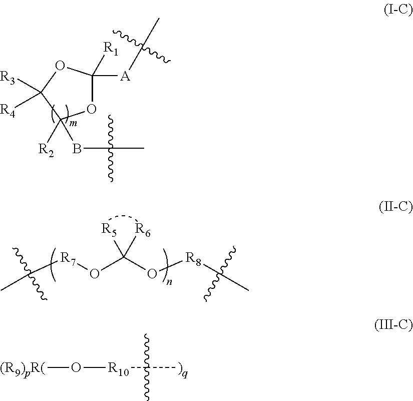

Similarly, the present invention also provides degradable cross-linked polymers each of which is made by polymerizing an isocyanate compound as described above with an epoxy resin and a degradable curing agent. The epoxy resin used can include a glycidyl ether epoxy resin, a glycidyl ester epoxy resin, glycidyl epoxy amine epoxy resin, a trifunctional epoxy resin, a tetrafunctional epoxy resin, a novolac epoxy resin, an o-cresol formaldehyde epoxy resin, an aliphatic epoxy resin, an alicyclic epoxy resin, or a nitrogen-containing epoxy resin; and the degradable curing agent can include an acetal or ketal aliphatic amine (as described in, e.g., WO 2012/071896, WO 2013007128, and CN 103249712A), an acetal or ketal aromatic amine or salt thereof (as described in, e.g., CN 103254406A), an acetal or ketal polyamine (as described in, e.g., CN 103012747A), a cyclic acetal or ketal amine (as described in, e.g., CN 103242509A), an acetal or ketal hydrazide (as described in, e.g., CN 103193959A), or hydrazine (as described in, e.g., CN 201310440092.0); and the cross-linked polymer has a cleavable cross-linking structure of Formula (I-C), Formula (II-C), or Formula (III-C), wherein R, R₁, R₂, R₃, R₄, A, B, R₅, R₆, R₇, R₈, m, and n, when present, are the same as those in the isocyanate compound.

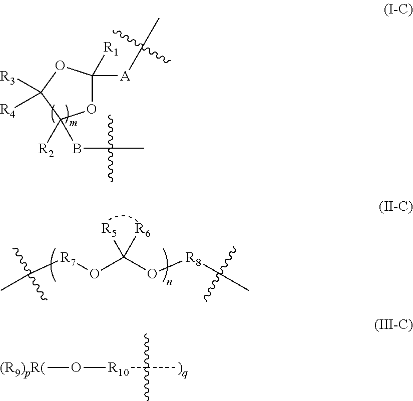

The present invention in yet another aspect further provides a method for degrading a polyurethane or a cross-linked polymer of this invention as just described above. Such a method includes at least the following steps: (1) under the heating and stirring conditions, the degradable polyurethane or the degradable cross-linked polymer is immersed in a mixed acid and solvent system for the degradation for 1˜600 hours at a temperature within the range of 15˜400° C., wherein the mass concentration of acid in the solvent 0.1˜99%; and (2) using an alkali solution to adjust the pH of the degradation solution to above 6 at a temperature within the range of 0˜200° C., wherein the mass concentration of alkali solution is 0.1˜99%.

In some embodiments of this method, the acid comprises hydrochloric acid, hydrobromic acid, hydrofluoric acid, acetic acid, trifluoroacetic acid, lactic acid, formic acid, propionic acid, citric acid, methanesulfonic acid, p-toluenesulfonic acid, nitric acid, sulfuric acid, sulfurous acid, phosphoric acid, perchloric acid, benzoic acid, salicylic acid, or phthalic acid; the solvent system comprises methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, dimethyl sulfoxide, tetrahydrofuran, methyl tetrahydrofuran, glycerol, or dioxane; the alkali comprises lithium hydroxide, sodium hydroxide, potassium hydroxide, calcium hydroxide, sodium carbonate, sodium bicarbonate, potassium carbonate, potassium bicarbonate, or ammonia; the solvent of the alkali solution comprises methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, dimethyl sulfoxide, tetrahydrofuran, methyl tetrahydrofuran, glycerol, or dioxane.

In some other embodiments of this method, in step (1), the heating temperature is within the range of 80˜150° C., the heating time is within the range of 4˜8 hours, and the mass concentration of acid in the solvent 0.5˜20%; in step (2), the temperature is within the range of 5˜50° C., the final pH value after adjustment with the alkali solution is in the range of 6˜12, and the mass concentration of alkali solution is within the range of 5˜30%.

Also provided by the present invention is a recyclable reinforced composite material which includes a polyurethane or a cross-linked polymer as just described above, a reinforcing material, and an auxiliary material. The reinforcing material can include, e.g., carbon nanotubes, boron nitride nanotubes, carbon black, metal nano-particles, metal oxide nanoparticles, organic nanoparticles, iron oxide, glass fibers, carbon fibers, natural fibers, synthetic fibers and fabric made therefrom; and the auxiliary material include, among others, an accelerator, a diluent, a plasticizer, a toughening agent, a thickening agent, a coupling agent, a defoamer, a flatting agent, an ultraviolet absorber, an antioxidant, a brightener, a fluorescent agent, a pigment, or a filler.

Also within the scope of the present invention is a method for recycling a reinforced composite material of claim 17, comprising the steps of: (1) under the heating and stirring conditions, immersing the reinforced composite material in a solution comprising an acid and a solvent and then heating the mixture at a temperature within the range of 15˜400° C. for 1˜600 hours to give rise to a degradation solution, wherein the mass concentration of acid in the solution is 0.1˜99%; (2) using an alkali solution of 0˜200° C. to adjust the pH value of the degradation solution from step (1) to be greater than 6 to obtain a precipitate, wherein the mass concentration of the alkali in the alkali solution is 0.1˜99%; and (3) separate, wash and dry the precipitate obtained in step (2).

In some embodiments, the acid comprises hydrochloric acid, hydrobromic acid, hydrofluoric acid, acetic acid, trifluoroacetic acid, lactic acid, formic acid, propionic acid, citric acid, methanesulfonic acid, p-toluenesulfonic acid, nitric acid, sulfuric acid, sulfurous acid, phosphoric acid, perchloric acid, benzoic acid, salicylic acid, or phthalic acid; the solvent comprises at least one of methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, dimethyl sulfoxide, tetrahydrofuran, methyl tetrahydrofuran, glycerol, or dioxane; the alkali comprises lithium hydroxide, sodium hydroxide, potassium hydroxide, calcium hydroxide, sodium carbonate, sodium bicarbonate, potassium carbonate, potassium bicarbonate, or ammonium hydroxide; and the alkali solvent comprises methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, dimethyl sulfoxide, tetrahydrofuran, methyl tetrahydrofuran, glycerol, or dioxane.

In still some other embodiments, in step (1), the mass concentration of acid in the solvent is within the range of 0.5˜20%, the temperature is within the range of 80˜200° C., and the reaction time is 2˜12 hours; and in step (2), the mass concentration of alkali solution is within the range of 5˜30%, the temperature is within the range of 5˜60° C.

A degradable isocyanate provided by the present invention and a macrodihydric alcohol can be used to prepare a degradable polyurethane matrix of this invention. Under the action of an acid, the cleavage or breaking of a particular chemical bond in the polyurethane results in the degradation of the polymer matrix. This degradation process may be performed under relatively mild, economical, and easily controlled reaction conditions. Therefore, the degradable polyurethanes of the present invention have significant environmental and economic advantages over conventional polyurathanes.

Due to their unique structure and excellent performance, polyurethane materials of polyfoam, elastomers, adhesives and others are widely used in construction, automotive, defense, aerospace and other fields. Currently, research of degradable polyurethanes is mostly focused on linear polyurethanes. However, these linear polyurethanes have poor mechanical properties and cannot undergo complete degradation. Degradable cross-linked polyurethanes of the present invention unexpectedly have much better mechanical properties and more complete degradation capability than linear polymers with a similar structure.

The egradable polyurethanes of this invention or polyurethane-modified epoxy resin can combine with degradable glass fibers, carbon fibers, natural fibers, synthetic fibers, or other fiber composite material to obtain the composite materials under the standard or common procedures of preparing composites materials. The composite materials can also be prepared by the combination of degradable polyurethane or polyurethane-modified epoxy resin with non-fibrous materials such as carbon nanotubes, boron nitride nanotubes, carbon black, metal nanoparticles, metal oxide nanoparticles, organic nanoparticles, iron oxide, or other non-fibrous materials.

A degradable composite material is typically degraded in the following manner: After a composite material is immersed in a hot recovery solution of acid and solvent, the polymer matrix would decompose first and then the reinforcing material can be separated and the polymer matrix can be recovered, e.g., after neutralizing the degradation solution with an alkaline solution to produce a precipitate. Under such conditions, the polymer matrix can be decomposed because it is an acid-sensitive cross-linked structure in which the bond cleavage of the acid-sensitive groups will occur. That will cause the crosslinked structure of the polymer matrix to be dissolved in a non-crosslinked polymer (e.g. a thermoplastic polymer) of an organic decomposition solvent. When the non-crosslinked polymer is fully dissolved, the reinforcing materials (e.g., carbon fibers) can be separated and removed from the degradation solution. The degradable polymer metrix yield can be recovered through the process of neutralization, sedimentation and solid-liquid separation. The reinforcing materials and recycled non-crosslinked polymers can therefore be separated, recovered and reused.

As discussed above, the present invention provides degradable polyurethanes and degradable corss-linked polymers—both of which are based on the degradable isocyanate compounds of this invention—and composite materials based on these degradable polyurethanes. Additionally, the present invention provides composite materials that can be prepared from degradable polyurethane-modified epoxy resin, curing agent, auxiliary materials and reinforcing materials (e.g., carbon fibers, glass fibers, synthetic fibers and natural fibers, etc.). Alternatively, the composite materials of this invention can be prepared from degradable polyurethane, auxiliary material and reinforced material. These composite materials can be degraded under relatively mild conditions, and 95% of reinforcing materials (e.g. carbon fibers, glass fibers, synthetic fibers and natural fibers, etc.) can be recovered to retain their original texture and the mechanical properties mostly, and they could be also reused, e.g., to prepare new composite materials. The recovered polymer matrix degradation products can be processed for the usage of plastic products. Such advantegous and efficient utilities of the degradable polyurethanes, degradable cross-linked polymers, and the composite materials made therefrom have never been reported, and the excellent efficiency, ease of handling and economic features of the recovery process also unexpectedly shown advantages over the conventional technologies and products.

The present invention illustrates that during the degradation process of polyurethane or polyurethane-modified epoxy resin composite material provided by the present invention, the cross-linked structure of polyurethane polymer matrix or polyurethane-modified epoxy resin composite material could be broken due to cleavage of specific chemical bonds, which leads to the degradation of the polymer matrix. And the cross-linked structure could be transformed into a non-crosslinked polymer (e.g. a thermoplastic polymer) that could be dissolved in an organic solvent. When the non-crosslinked polymer is fully dissolved in an organic solvent, the reinforced materials can be removed from the solution thereby recovered for potential reuse. The degradation product of the polymer metrix can be recovered through the process of neutralization, sedimentation and solid-liquid separation. The reinforcing materials and recycled non-crosslinked polymers can also be separated, recovered and reused.

The currently composites recovery technology requires incinerating the plastic component of a composite to recover the reinforcing material. However, as a significant advantage, the plastic component of degradable composite materials of this invention and the reinforcing materials used to make the composite materials can both be recycled more efficiently. For instance, the cross-linked polymers of this invention could be degraded to form a thermoplastic polymer wherein, after a small amount of acetal matrix has been lost, the mass recovery ratio of the thermoplastic polymer is high and this polymer can be processed for the industrial use. Also, the mass recovery ratio of the cross-linked polymer of this invention and reinforcing material used therein is greater than 96%. Under the acid recycling conditions, the recycled reinforcement has the stable property with the clean surface and no defects. The recovery methods of the degradable composites of this invention can be carried out under mild conditions, and are economic and easy to control.

As used herein, the term “alkyl,” when used alone or as part of a larger moiety (e.g., as in “alkyl-hetero-alkyl”), refers to a saturated aliphatic hydrocarbon group. It can contain 1 to 12 (e.g., 1 to 8, 1 to 6, or 1 to 4) carbon atoms. As a moiety, it can be denoted as —CₙH₂ₙ₊₁. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, and 2-ethylhexyl. An alkyl group can be substituted (i.e., optionally substituted) with one or more substituents. When an alkyl is preceded by a carbon-number modifier, e.g., C₁₋₈, its means the alkyl group contains 1 to 8 carbon atoms.

As used herein, the term “alkylene,” when used alone or as part of a larger moiety (e.g., as in “alkylene-oxy-hetero-cyclic”), refers to a saturated aliphatic hydrocarbon group with two radical points for forming two covalent bonds with two other moieties. It can contain 1 to 12 (e.g., 1 to 8, 1 to 6, or 1 to 4) carbon atoms. As a moiety, it can be denoted as —CₙH₂ₙ—. Examples of an alkylene group include, but are not limited to, methylene (—CH₂—), ethylene (—CH₂CH₂—), and propylene (—CH₂CH₂CH₂—). When an alkylene is preceded by a carbon-number modifier, e.g., C₂₋₈, it means the alkylene group contains 2 to 8 carbon atoms.

As used herein, the term “alkynyl,” when used alone or as part of a larger moiety, refers to an aliphatic hydrocarbon group with at least one triple bond. It can contain 2 to 12 (e.g., 2 to 8, 2 to 6, or 2 to 4) carbon atoms. An alkynyl group can be straight or branched. Examples of an alkynyl group include, but are not limited to, propargyl and butynyl. When an alkynyl is preceded by a carbon-number modifier, e.g., C₂₋₈, it means the alkynyl group contains 2 to 8 carbon atoms.

As used herein, the term “alkenyl,” when used alone or as part of a larger moiety, refers to an aliphatic hydrocarbon group with at least one double bond. It can contain 2 to 12 (e.g., 2 to 8, 2 to 6, or 2 to 4) carbon atoms. An alkenyl group with one double bond can be denoted as —CₙH₂ₙ₋₁, or —CₙH₂ₙ₋₃ with two double bonds. Like an alkyl group, an alkenyl group can be straight or branched. Examples of an alkenyl group include, but are not limited to, allyl, isoprenyl, 2-butenyl, and 2-hexenyl. When an alkylene is preceded by a carbon-number modifier, e.g., C₃₋₈, it means the alkylene group contains 3 to 8 carbon atoms.

As used herein, the term “cycloalkyl,” when used alone or as part of a larger moiety (e.g., as in “oxy-cycloalkyl”), refers to a saturated carbocyclic mono-, bi-, or tri-cyclic (fused or bridged or spiral) ring system. It can contain 3 to 12 (e.g., 3 to 10, or 5 to 10) carbon atoms. Examples of cycloalkyl groups include, but are not limited to, cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, ((aminocarbonyl)cycloalkyl)cycloalkyl. When a cycloalkyl is preceded by a carbon-number modifier, e.g., C₃₋₈, its means the alkyl group contains 3 to 8 carbon atoms.

As used herein, the term “cycloalkenyl,” when used alone or as part of a larger moiety (e.g., as in “oxy-cycloalkenyl”), refers to a non-aromatic carbocyclic ring system having one or more double bonds. It can contain 3 to 12 (e.g., 3 to 10, or 5 to 10) carbon atoms. Examples of cycloalkenyl groups include, but are not limited to, cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, orbicyclo[3.3.1]nonenyl.

As used herein, the term “heterocycloalkyl,” when used alone or as part of a larger moiety (e.g., as in “cycloalkylene-oxy-cycloalkenyl”), refers to a 3- to 16-membered mono-, bi-, or tri-cyclic (fused or bridged or spiral)) saturated ring structure, in which one or more of the ring atoms is a heteroatom (e.g., N, O, S, or combinations thereof). In addition to the heteroatom(s), the heterocycloalkyl can contain 3 to 15 carbon atoms (e.g., 3 to 12 or 5 to 10). Examples of a heterocycloalkyl group include, but are not limited to, piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, I-aza-bicyclo[2.2.2]octyl, 3-aza-bicyclo[3.2.1]octyl, and 2,6-dioxa-tricyclo[3.3.1.03,7]nonyl. A monocyclic heterocycloalkyl group can be fused with a phenyl moiety such as tetrahydroisoquinoline. When a heterocycloalkyl is preceded by a carbon-number modifier, e.g., C₄₋₈, it means the heterocycloalkyl group contains 4 to 8 carbon atoms.

As used herein, the term “hetero,” when used alone or as part of a larger moiety (e.g., as in “heterocyclo,” “heterocycloalkyl,” “heterocycloalkylene” or “heteroaryl”), refers to a hetero atom or group that is —O—, —S—, or —NH—, if applicable.

As used herein, the term “aryl,” when used alone or as part of a larger moiety (e.g., as in “alkylenearyl”), refers to a monocyclic (e.g., phenyl), bicyclic (e.g., indenyl, naphthalenyl, or tetrahydronaphthyl), and tricyclic (e.g., fluorenyl, tetrahydrofluorenyl, tetrahydroanthracenyl, or anthracenyl) ring system in which the monocyclic ring system is aromatic (e.g., phenyl) or at least one of the rings in a bicyclic or tricyclic ring system is aromatic (e.g., phenyl). The bicyclic and tricyclic groups include, but are not limited to, benzo-fused 2- or 3-membered carbocyclic rings. For instance, a benzo-fused group includes phenyl fused with two or more C₄₋₈ carbocyclic moieties.

As used herein, the term “heteroaryl” refers to a monocyclic, bicyclic, or tricyclic ring system having 5 to 15 ring atoms wherein at least one of the ring atoms is a heteroatom (e.g., N, O, S, or combinations thereof) and when the monocyclic ring system is aromatic or at least one of the rings in the bicyclic or tricyclic ring systems is aromatic. It can contain 5 to 12 or 8 to 10 ring atoms. A heteroaryl group includes, but is not limited to, a benzo-fused ring system having 2 to 3 rings. For example, a benzo-fused group includes benzo fused with one or two 4- to 8-membered heterocycloalkyl moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are pyridyl, IH-indazolyl, furyl, pyrrolyl, thienyl, thiazolyl, oxazolyl, imidazolyl, tetrazolyl, benzofuryl, isoquinolinyl, benzithiazolyl, xanthenyl, thioxanthenyl, phenothiazinyl, dihydroindolyl, benzo[I,3]dioxolyl, benzo [b]furyl, benzo [bjthiophenyl, indazolyl, benzimidazolyl, benzthiazolyl, puryl, quinolinyl, quinazolinyl, phthalazyl, quinazolyl, quinoxalyl, isoquinolinyl, 4H-quinolizyl, benzo-1,2,5-thiadiazolyl, and 1,8-naphthyridyl.

As used herein, the suffix “-ene” is used to describe a bivalent group with two radical points for forming two covalent bonds with two other moieties. In other words, any of the terms as defined above can be modified with the suffix “-ene” to describe a bivalent version of that moiety. For example, a bivalent aryl ring structure is “arylene,” a bivalent benzene ring structure is “phenylene,” a bivalent heteroaryl ring structure is “heteroarylene,” a bivalent cycloalkyl ring structure is a “cycloalkylene,” a bivalent heterocycloalkyl ring structure is “heterocycloalkylene,” a bivalent cycloalkenyl ring structure is “cycloalkenylene,”

As used herein, the term “optionally” (e.g., as in “optionally substituted with”) means that the moiety at issue is either substituted or not substituted, and that the substitution occurs only when it is chemically feasible. For instance, H cannot be substituted with a substituent and a covalent bond or —C(═O)— group cannot be substituted with a substituent.

As used herein, an “oxo” or “oxide” group refers to ═O.

As used herein, an “oxy” group refers to —O—.

As used herein, a “carbonyl” group refers to —C(O)— or —C(═O)—.

As used herein, the term “1,4-alkyl substituted piperazine” refers to

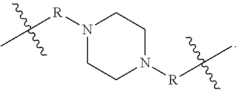

For convenience and as commonly understood, the term “optionally substituted” only applies to the chemical entities that can be substituted with suitable substituents, not to those that cannot be substituted chemically.

As used herein, the term “or” can mean “or” or “and.”

### DETAILED DESCRIPTION OF THE INVENTION

### Example 1

### Synthesis of Bis(4-Isocyanatophenoxy)Methane (“Isocyanate Compound 1”)

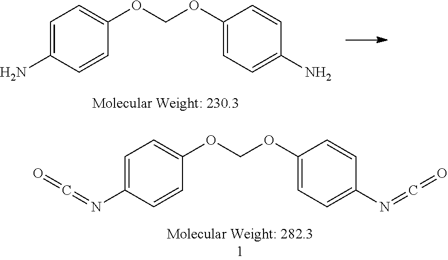 [SMILES: Nc1ccc(OCOc2ccc(N)cc2)cc1 ; O=C=Nc1ccc(OCOc2ccc(N=C=O)cc2)cc1]

A solution of 4,4′-(methylenebis(oxy)) dianiline (23 g) in 200 mL dioxane was slowly added into a solution of triphosgene (29.6 g) in 300 mL toluene, with the temperature controlled below 5° C. On the completion of the addition, the resultant mixture was refluxed for 4 hours. The reaction mixture was then concentrated in vacuo, and the residue was suspended in 500 mL petroleum ether with vigorous stirring. The mixture was filtered. The filtrate was dried with anhydrous Na₂SO₄, and concentrated in vacuo giving 15 g of Isocyanate Compound 1 as white solid with the yield of 53.2%. ¹H-NMR (CDCl₃, 400 M): 5.65 (2H, s), 7.02 (8H, s).

### Example 2

### Synthesis of Isocyanate Compound 1

A solution of 4,4′-(methylenebis(oxy)) dianiline (23 g) in 450 mL ethyl acetate was slowly added into a solution of triphosgene (29.6 g) in 150 mL ethyl acetate, with the temperature controlled below 30° C. On the completion of the addition, the resultant mixture was refluxed overnight. The reaction mixture was then concentrated in vacuo, and the residue was suspended in 500 mL petroleum ether with vigorous stirring. The mixture was filtered. The filtrate was dried with anhydrous Na₂SO₄, and concentrated in vacuo giving 18 g of Isocyanate Compound 1 as a white solid with the yield of 63.8%. ¹H-NMR (CDCl₃, 400 M): 5.65 (2H, s), 7.02 (8H, s).

### Example 3

### Synthesis of Isocyanate Compound 1

A solution of 4,4′-(methylenebis(oxy)) dianiline (2.3 g) in 20 mL dichloromethane was slowly added into a solution of triphosgene (5.92 g) in 40 mL dichloromethane, followed with a mixture of 6 mL triethylamine and 20 mL dichloromethane. During the addition processes, the temperature gradually increased, and the reaction mixture started refluxing. On the completion of the addition, the resultant mixture was stirred at room temperature for 30 mins, and then concentrated in vacuo. The residue was suspended in 300 mL petroleum ether with vigorous stirring. The mixture was filtered. The filtrate was dried with anhydrous Na₂SO₄, and concentrated in vacuo giving 1.3 g of Isocyanate Compound 1 as a white solid with the yield of 46.1%. ¹H-NMR (CDCl₃, 400 M): 5.65 (2H, s), 7.02 (8H, s).

### Example 4

### Synthesis of Isocyanate Compound 1

At 0° C., a solution of 4,4′-(methylenebis(oxy)) dianiline (2.30 g) in 20 mL dioxane was slowly added into a solution of triphosgene (2.96 g) in 30 mL dioxane, with the temperature of the mixture controlled below 30° C. Upon the completion of the addition, the resultant mixture was refluxed overnight. The reaction mixture was then concentrated in vacuo, and the residue was suspended in 100 mL petroleum ether with vigorous stirring. After filtering the mixture, the filtrate was dried with anhydrous Na₂SO₄ and concentrated in vacuoto give 1.2 g of Isocyanate Compound 1 as a white solid at the yield of 42.5%. ¹H-NMR (CDCl₃, 400 M): 5.65 (2H, s), 7.02 (8H, s).

### Example 5

### Synthesis of Bis(2-Isocyanatoethoxy)Methane (Isocyanate Compound 2)

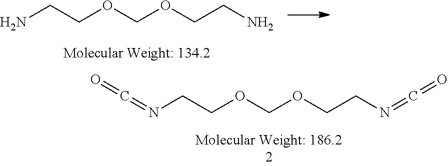 [SMILES: NCCOCOCCN ; O=C=NCCOCOCCN=C=O]

At the room temperature, a solution of 2,2′-(methylenebis(oxy)) diethanamine (1.34 g) in 10 mL dichloromethane was slowly added into a solution of triphosgene (5.92 g) in 40 mL dichloromethane, followed with a mixture of 6 mL triethylamine and 20 mL dichloromethane. During the above addition processes, the temperature was gradually increased, and the reaction mixture started refluxing. Upon the completion of the addition, the resultant mixture was stirred at the room temperature for 30 minutes. The resultant reaction mixture was concentrated in vacuo, and the residue was suspended in 100 mL petroleum ether with vigorous stirring. The mixture was filtered and the filtrate was dried with anhydrous Na₂SO₄ and concentrated in vacuoto give 1.1 g of Isocyanate Compound 2 a slight yellow oil at the yield of 60%. ¹H-NMR (CDCl₃, 400 M) 3.50 (2H, t), 3.79 (2H, t), 4.70 (2H, s).

### Example 6

### Synthesis of Isocyanate Compound 2

At the room temperature, a solution of 2,2′-(methylenebis(oxy)) diethanamine (1.34 g) and N, N-diisopropylethylamine (1.07 mL) in 10 mL dichloromethane was slowly added into a solution of triphosgene (2.96 g) in 20 mL dichloromethane. Upon the completion of the addition, the resultant mixture was stirred at room temperature for 30 mins. The resultant reaction mixture was concentrated in vacuo, and the residue was suspended in 100 mL petroleum ether with vigorous stirring. Then the mixture was filtered and the filtrate was dried with anhydrous Na₂SO₄, and concentrated in vacuoto give 1.0 g of Isocyanate Compound 2 as light yellow oil with the yield of 53.7%. ¹H-NMR (CDCl₃, 400 M) 3.50 (2H, t), 3.79 (2H, t), 4.70 (2H, s).

### Example 7

### Synthesis of Isocyanate Compound 2

At 0° C., a solution of 2,2′-(methylenebis(oxy)) diethanamine (1.34 g) and pyridine (1.1 mL) in 10 mL dichloromethane was slowly added into a solution of triphosgene (2.96 g) in 20 mL dichloromethane, with the temperature controlled below 5° C. Upon the completion of the addition, the resultant mixture was stirred at the room temperature for 30 minutes. The resultant reaction mixture was concentrated in vacuo, and the residue was suspended in 100 mL petroleum ether with vigorous stirring. The mixture was filtered. The filtrate was dried with anhydrous Na₂SO₄, and concentrated in vacuo giving 1.0 g of Isocyanate Compound 2 as light yellow oil with the yield of 53.7%. ¹H-NMR (CDCl₃, 400 M) 3.50 (2H, t), 3.79 (2H, t), 4.70 (2H, s).

### Example 8

### Synthesis of Isocyanate Compound 2

At the room temperature, a solution of 2,2′-(methylenebis(oxy)) diethanamine (13.4 g) in 150 mL ethyl acetate was slowly added into a solution of triphosgene (29.6 g) in 150 mL ethyl acetate, with the temperature controlled below 30° C. On the completion of the addition, the resultant mixture was refluxed overnight. The resultant reaction mixture was concentrated in vacuo, and the residue was suspended in 500 mL petroleum ether with vigorous stirring. The mixture was filtered. The filtrate was dried with anhydrous Na₂SO₄, and concentrated in vacuo giving 12 g of Isocyanate Compound 2 as light yellow oil with the yield of 63.8%. ¹H-NMR (CDCl₃, 400 M) 3.50 (2H, t), 3.79 (2H, t), 4.70 (2H, s).

### Example 9

### Synthesis of Isocyanate Compound 3

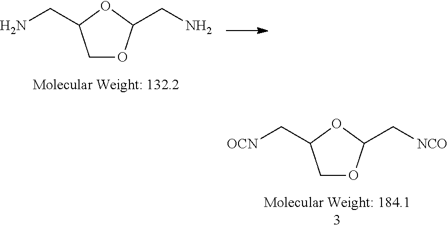

At the room temperature, a solution of (1,3-dioxolane-2,4-diyl)dimethanamine (2 g) in 10 mL dichloromethane was slowly added into a solution of triphosgene (10 g) in 80 mL dichloromethane, followed with a mixture of 6 mL of triethylamine and 20 mL of dichloromethane. During the above addition processes, the temperature gradually increased, and the reaction mixture started refluxing. On the completion of the addition, the resultant mixture was stirred at room temperature overnight. The resultant reaction mixture was concentrated in vacuo, and the residue was suspended in 100 mL petroleum ether with vigorous stirring. The mixture was filtered, and the filtrate was dried with anhydrous Na₂SO₄ and then concentrated in vacuoto give 1.7 g of Isocyanate Compound 3 as light yellow oil with the yield of 61%.

### Example 10

### Preparation of Degradable Polyurethane

Polyethyleneglycol 1000 and Isocyanate Compound 1 were mixed at the mass ratio of 100/28.2. After quickly defoamed under vacuum with vigorous stirring, the mixture was cured at the room temperature, followed by postcured in an 80° C. oven for 2 hours to give a degradable polyurethane.

### Example 11

### Preparation of Degradable Polyurethane

Polyethyleneglycol 1000 and Isocyanate Compound 2 were mixed at the mass ratio of 100/18.6. After quickly defoamed under vacuum with vigorous stirring, the mixture was cured at the room temperature, followed by postcured in an 80° C. oven for 2 hours to give a degradable polyurethane.

### Example 12

### Preparation of Degradble Polyurethane

Polyethyleneglycol 1000 and Isocyanate Compound 3 were mixed at the mass ratio of 100/18.4. After quickly defoamed under vacuum with vigorous stirring, the mixture was cured at room temperature, followed by postcured in an 80° C. oven for 2 hours to give a degradable polyurethane.

### Example 13

### Degradation of Degradable Polyurethane

In a round-bottomed flask, a piece of the degradable polyurethane sample (1.0 g) from Example 10 was imerged in a mixture of 10 mL concentrated hydrochloric acid and 90 mL ethylene glycol. The degradation solution was stirred at 100° C. for 4 hours to give a clear solution which was neutralized with a 20% aqueous sodium hydroxide solution. The resultant suspension was filtered, and the collected solid was washed with water and dried, giving a mass recovery yield of 96.5%.

### Example 14

### Degradation of Degradable Polyurethane

In a round-bottomed flask, a piece of the degradable polyurethane sample (1.0 g) from Example 10 was imerged in a mixture of 1 mL concentrated hydrochloric acid and 90 mL ethylene glycol. The degradation solution was stirred at 180° C. for 2 hours to give a clear solution which was neutralized with a 20% aqueous sodium hydroxide solution. The resultant suspension was filtered, and the collected solid was washed with water and dried, giving a mass recovery yield of 96%.

### Example 15

### Degradation of Degradable Polyurethane

In a round-bottomed flask, a piece of the degradable polyurethane sample (1.0 g) from Example 11 was imerged in a mixture of 10 mL concentrated hydrochloric acid and 90 mL ethylene glycol. The degradation solution was stirred at 100° C. for 4 hours giving a clear solution which was neutralized with a 20% aqueous sodium hydroxide solution. The resultant suspension was filtered, and the collected solid was washed with water and dried, giving a mass recovery yield of 97%.

### Example 16

### Degradation of Degradable Polyurethane

In a round-bottomed flask, a piece of degradable polyurethane sample (1.0 g) from Example 11 was imerged in a mixture of 1 mL concentrated hydrochloric acid and 90 mL ethylene glycol. The degradation solution was stirred at 180° C. for 2 hours giving a clear solution which was neutralized with a 20% aqueous sodium hydroxide solution. The resultant suspension was filtered, and the collected solid was washed with water and dried, giving a mass recovery yield of 98%.

### Example 17

### Degradation of Degradable Polyurethane

In a round-bottomed flask, a piece of the degradable polyurethane sample (1.0 g) from Example 12 was imerged in a mixture of 1 mL concentrated hydrochloric acid and 90 mL ethylene glycol. The degradation solution was stirred at 140° C. for 1 hour to give a clear solution which was neutralized with a 20% aqueous sodium hydroxide solution. The resultant suspension was filtered, and the collected solid was washed with water and dried, giving a mass recovery yield of 98%.

### Example 18

### Preparation of Recyclable Carbon Fibre Polyurethane Composite

Polyethyleneglycol 1000 and Isocyanate Compound 1 were mixed at the mass ratio of 100/28.2. After quickly defoamed under vacuum with vigorous stirring, the mixture was evenly applied over three layers of 2×2 twill carbon fiber (3K) fabric sheets. The resultant stack was then cured on a flat hot-pressing machine at 80° C. under a pressure of 10 atms for 2 hours, giving a recyclable carbon fiber polyurethane composite laminate.

### Example 19

### Preparation of Recyclable Carbon Fibre Polyurethane Composite

Polyethyleneglycol 1000 and isocyanate II were mixed at the mass ratio of 100/18.6. After quickly defoamed under vacuum with vigorous stirring, the mixture was evenly applied over three layers of 2×2 twill carbon fiber (3K) fabric sheets. The resultant stack was then cured on a flat hot-pressing machine at 80° C. under a pressure of 10 atms for 2 hours, giving a recyclable carbon fiber polyurethane composite laminate.

### Example 20

### Preparation of Recyclable Carbon Fibre Polyurethane Composite

Polyethyleneglycol 1000 and isocyanate III were mixed at the mass ratio of 100/18.4. After quickly defoamed under vacuum with vigorous stirring, the mixture was evenly applied over three layers of 2×2 twill carbon fiber (3K) fabric sheets. The resultant stack was then cured on a flat hot-pressing machine at 80° C. under a pressure of 10 atms for 2 hours giving a recyclable carbon fiber polyurethane composite laminate.

### Example 21

### Degradation of Degradable Polyurethane in Recyclable Carbon Fiber Polyurethane Composite Panel

In a round-bottomed flask, a piece of recyclable carbon fiber polyurethane composite sample (1.0 g) from Example 18 was imerged in a mixture of 10 mL concentrated hydrochloric acid and 90 mL ethylene glycol. After heated at 100° C. for 4 hours, the degradation solution was filtered to separate the carbon fibres, and the filtrate was neutralized with a 20% aqueous sodium hydroxide solution. The resultant suspension was filtered again, and the collected solid was washed with water and dried, giving a mass recovery yield of 96%.

### Example 22

### Degradation of Degradable Polyurethane in Recyclable Carbon Fiber Polyurethane Composite Panel

In a round-bottomed flask, a piece of recyclable carbon fiber polyurethane composite sample (1.0 g) from Example 18 was imerged in a mixture of 1 mL concentrated hydrochloric acid and 90 mL ethylene glycol. After heated at 180° C. for 2 hours, the degradation solution was filtered to separate the carbon fibres, and the filtrate was neutralized with a 20% aqueous sodium hydroxide solution. The resultant suspension was filtered again, and the collected solid was washed with water and dried, giving a mass recovery yield of 96%.

### Example 23

### Degradation of Degradable Polyurethane in Recyclable Carbon Fiber Polyurethane Composite Panel

In a round-bottomed flask, a piece of recyclable carbon fiber polyurethane composite sample (1.0 g) from Example 19 was imerged in a mixture of 10 mL concentrated hydrochloric acid and 90 mL ethylene glycol. After heated at 120° C. for 2 hours, the degradation solution was filtered to separate the carbon fibres, and the filtrate was neutralized with a 20% aqueous sodium hydroxide solution. The resultant suspension was filtered again, and the collected solid was washed with water and dried, giving a mass recovery yield of 97%.

### Example 24

### Degradation of Degradable Polyurethane in Recyclable Carbon Fiber Polyurethane Composite Panel

In a round-bottomed flask, a piece of recyclable carbon fiber polyurethane composite sample (1.0 g) from Example 19 was imerged in a mixture of 5 mL concentrated hydrochloric acid and 90 mL ethylene glycol. After heated at 150° C. for 4 hours, the degradation solution was filtered to separate the carbon fibres, and the filtrate was neutralized with a 20% aqueous sodium hydroxide solution. The resultant suspension was filtered again, and the collected solid was washed with water and dried, giving a mass recovery yield of 98%.

### Example 25

### Degradation of Degradable Polyurethane in Recyclable Carbon Fiber Polyurethane Composite Panel

In a round-bottomed flask, a piece of recyclable carbon fiber polyurethane composite sample (1.0 g) from Example 20 was imerged in a mixture of 5 mL concentrated hydrochloric acid and 90 mL ethylene glycol. After heated at 160° C. for 6 hours, the degradation solution was filtered to separate the carbon fibres, and the filtrate was neutralized with a 20% aqueous sodium hydroxide solution. The resultant suspension was filtered again, and the collected solid was washed with water and dried, giving a mass recovery yield of 97%.

### Example 26

### Degradable Cross-Linked Polymer

In a round-bottomed flask, degradable Isocyanate Compound 1 (2.8 g) and liquid bisphenol A type epoxy resin E52D (EEW 188.5 g/eq, 18.9 g) were stirred at 120° C. for 2 hours. The resultant product was then mixed with degradable curing agent 2,2′-(methylenebis(oxy)) diethanamine (AEW 33.5 g/eq, 3.4 g) at room temperature, and the mixture was cured at 120° C. for 2 hours giving the degradable polymer.

### Example 27

### Degradable Polymer

At the room temperature and in a round-bottomed flask, degradable Isocyanate Compound 2 (2.8 g), liquid bisphenol A type epoxy resin E52D (EEW 188.5 g/eq, 18.9 g), and degradable curing agent 4,4′-(methylenebis(oxy)) dianiline (AEW 57.5 g/eq, 5.8 g) were mixed and stirred. The resultant mixture was then spreaded on a panel and cured at 120° C. for 2 hours, giving a degradable cross-linked polymer.

### Example 28

### Degradable Polymer

At the room temperature and in a round-bottomed flask, degradable isocyanate III (2.7 g), liquid bisphenol A type epoxy resin E52D (EEW 188.5 g/eq, 18.9 g), and degradable curing agent 4,4′-(methylenebis(oxy)) dianiline (AEW 57.5 g/eq, 5.8 g). The resultant mixture was then spreaded on a panel and cured at 120° C. for 2 hours giving the degradable polymer.

### Example 29

### Degradation of the Degradable Polymer

In a round-bottomed flask, a piece of degradable polmer sample (0.5 g) from Example 26 was imerged in a mixture of 10 mL concentrated hydrochloric acid and 90 mL ethylene glycol. The degradation solution was stirred at 180° C. for 10 hours giving a clear solution which was neutralized with a 20% aqueous sodium hydroxide solution. The resultant suspension was filtered, and the collected solid was washed with water and dried giving 0.48 g of degraded polymer of mass recovery yield of 96%.

### Example 30

### Degradation of the Degradable Polymer

In anauotclave, a piece of degradable polmer sample (0.7 g) from Example 26 was treated in a mixture of 0.1 mL concentrated hydrochloric acid and 90 mL ethylene glycol. The degradation solution was stirred at 350° C. for 0.5 hours. The resultant clear solution was transferred into a clean beaker, and neutralized with a 20% aqueous sodium hydroxide solution. The resultant suspension was filtered, and the collected solid was washed with water and dried giving 0.66 g of degraded polymer of mass recovery yield of 94%.

### Example 31

### Degradation of the Degradable Polymer

In a round-bottomed flask, a piece of degradable polmer sample (0.4 g) from Example 26 was imerged in a mixture of 30 mL concentrated hydrochloric acid and 70 mL ethylene glycol. The degradation solution was stirred at room temperature for 120 hours giving a clear solution which was neutralized with a 20% aqueous sodium hydroxide solution. The resultant suspension was filtered, and the collected solid was washed with water and dried giving 0.38 g of degraded polymer of mass recovery yield of 95%.

### Example 32

### Degradation of the Degradable Polymer

In a round-bottomed flask, a piece of degradable polmer sample (0.7 g) from Example 27 was imerged in a mixture of 10 mL concentrated hydrochloric acid and 90 mL ethylene glycol. The degradation solution was stirred at 190° C. for 4 hours giving a clear solution which was neutralized with a 20% aqueous sodium hydroxide solution. The resultant suspension was filtered, and the collected solid was washed with water and dried giving 0.68 g of degraded polymer of mass recovery yield of 97%.

### Example 33

### Degradation of the Degradable Polymer

In a round-bottomed flask, a piece of degradable polmer sample (0.6 g) from Example 27 was imerged in a mixture of 10 mL concentrated hydrochloric acid and 45 mL benzyl alcohol. The degradation solution was stirred at 190° C. for 4 hours giving a clear solution which was neutralized with a 20% aqueous sodium hydroxide solution. The resultant suspension was filtered, and the collected solid was washed with water and dried giving 0.57 g of degraded polymer of mass recovery yield of 95%.

### Example 34

### Degradation of the Degradable Polymer

In a round-bottomed flask, a piece of degradable polmer sample (0.6 g) from Example 28 was imerged in a mixture of 10 mL concentrated hydrochloric acid and 90 mL octanol. The degradation solution was stirred at 120° C. for 8 hours giving a clear solution which was neutralized with a 20% aqueous sodium hydroxide solution. The resultant suspension was filtered, and the collected solid was washed with water and dried giving 0.58 g of degraded polymer of mass recovery yield of 96%.

### Example 35

### Degradation of the Degradable Polymer

In a round-bottomed flask, a piece of degradable polmer sample (0.63 g) from Example 28 was imerged in a mixture of 10 mL concentrated hydrochloric acid and 90 mL octanol. The degradation solution was stirred at 155° C. for 4 hours giving a clear solution which was neutralized with a 20% aqueous sodium hydroxide solution. The resultant suspension was filtered, and the collected solid was washed with water and dried giving 0.6 g of degraded polymer of mass recovery yield of 95%.

### Example 36

### Preparation Of Recyclable Carbon Fiber Composite Laminate

At the room temperature and in a round-bottomed flask, degradable Isocyanate Compound 1 (28 g) and liquid bisphenol A type epoxy resin E52D (EEW 188.5 g/eq, 189 g) were stirred at 120° C. for 2 hours. The resultant product was further mixed with degradable curing agent 2,2′-(methylenebis(oxy)) diethanamine (AEW 33.5 g/eq, 34 g) at the room temperature, and the mixture was then quickly defoamed under vacuum with vigorous stirring. It was then coiled to the room temperature and then stored in a freezer.

After being heated to 50° C., the resultant resin mixture was evenly applied over five layers of 2×2 twill carbon fiber (3K) fabric sheets. And the stack was then cured on a flat hot-pressing machine at 150° C. under a pressure of 10 atms for 2 hours, giving a recyclable carbon fiber composite laminate.

### Example 37

### Preparation for Recyclable Carbon Fiber Composite Laminate

At the room temperature and in a round-bottomed flask, degradable Isocyanate Compound 2 (28 g), liquid bisphenol A type epoxy resin E52D (EEW 188.5 g/eq, 189 g) were stirred at 120° C. for 2 hours. The resultant product was further mixed with degradable curing agent 4,4′-(methylenebis(oxy)) dianiline (AEW 57.5 g/eq, 58 g) at the room temperature on a three roll grinder, and the mixture was then quickly defoamed under vacuum in a high speed mixer, cooled to the room temperature and then stored in a freezer.

Carbon fibre prepreg was prepared by impregnating 2×2 twill carbon fiber (3K) fabric sheet with the above prepared degradable epoxyresin at 50° C. A stack of five layers of the prepared carbon fiber prepreg was cured on a flat hot-pressing machine at 150° C. under a pressure of 10 atms for 2 hours, giving a recyclable carbon fiber composite laminate.

### Example 38

### Preparation for Recyclable Carbon Fiber Composite Laminate

At the room temperature and in a round-bottomed flask, degradable Isocyanate Compound 3 (27.8 g) and liquid bisphenol A type epoxy resin E52D (EEW 188.5 g/eq, 189 g) were stirred at 120° C. for 2 hours. The resultant product was further mixed with degradable curing agent 4,4′-(methylenebis(oxy)) dianiline (AEW 57.5 g/eq, 58 g) at the room temperature on a three roll grinder, and the mixture was then quickly defoamed under vacuum in a high speed mixer and stored in a freezer.

Carbon fibre prepreg was prepared by impregnating 2×2 twill carbon fiber (3K) fabric sheet with the above prepared degradable epoxyresin at 50° C. A stack of five layers of the prepared carbon fiber prepreg was cured on a flat hot-pressing machine at 150° C. under a pressure of 10 atms for 2 hours giving a recyclable carbon fiber composite laminate.

### Example 39

### Degradation of Recyclable Carbon Fiber Composite Laminate

In a round-bottomed flask, a piece of recyclable carbon fiber composite laminate sample (1.6 g) from Example 36 was imerged in a mixture of 10 mL concentrated hydrochloric acid and 90 mL benzyl alcohol. After heated at 190° C. for 3 hours, the degradation solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 1.55 g, giving a mass recovery yield of 97%. The surface of the recycled carbon fiber was very clean and did not have any sign of damage.

### Example 40

### Degradation of Recyclable Carbon Fiber Composite Laminate

In a round-bottomed flask, a piece of recyclable carbon fiber composite laminate sample (1.5 g) from Example 36 was imerged in a mixture of 10 mL concentrated hydrochloric acid and 90 mL ethylene glycol. After heated at 160° C. for 3 hours, the degradation was almost complete and the solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 1.46 g, giving a mass recovery yield of 97%. The surface of the recycled carbon fiber was very clean and essentially free of signs of damage.

### Example 41

### Degradation of Recyclable Carbon Fiber Composite Laminate

In a round-bottomed flask, a piece of recyclable carbon fiber composite laminate sample (1.3 g) from Example 36 was imerged in a mixture of 10 mL concentrated hydrochloric acid and 90 mL hexanol. After heated at 135° C. for 4 hours, the degradation was almost complete and the solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 1.23 g, giving a mass recovery yield of 95%. The surface of the recycled carbon fiber was very clean and essentially free of signs of damage.

### Example 42

### Degradation of Recyclable Carbon Fiber Composite Laminate

In a round-bottomed flask, a piece of recyclable carbon fiber composite laminate sample (1.0 g) from Example 36 was imerged in a mixture of 10 mL concentrated hydrochloric acid and 90 mL octanol. After heated at 135° C. for 4 hours, the degradation was almost complete and the solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 0.96 g, giving a mass recovery yield of 96%. The surface of the recycled carbon fiber was very clean and essentially free of signs of damage.

### Example 43

### Degradation of Cyclable Carbon Fiber Composite Laminate

In a round-bottomed flask, a piece of recyclable carbon fiber composite laminate sample (0.9 g) from Example 37 was imerged in a mixture of 10 mL concentrated hydrochloric acid and 90 mL ethylene glycol. After heated at 135° C. for 4 hours, the degradation was almost complete and the solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 0.86 g, giving a mass recovery yield of 95%. The surface of the recycled carbon fiber was very clean and essentially free of signs of damage.

### Example 44

### Degradation of Recyclable Carbon Fiber Composite Laminate

In a round-bottomed flask, a piece of recyclable carbon fiber composite laminate sample (1.1 g) from Example 37 was imerged in a mixture of 5 mL concentrated hydrochloric acid and 90 mL ethylene glycol. After heated at 185° C. for 3 hours, the degradation was almost complete and the solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 0.86 g, giving a mass recovery yield of 95%.

### Example 45

### Degradation of Recyclable Carbon Fiber Composite Laminate

In a round-bottomed flask, a piece of recyclable carbon fiber composite laminate sample (2 g) from Example 37 was imerged in a mixture of 5 mL methanesulfonic acid and 90 mL octanol. After heated at 160° C. for 3 hours, the degradation was almost complete and the solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 1.94 g, giving a mass recovery yield of 97%. The surface of the recycled carbon fiber was very clean and essentially free of signs of damage.

### Example 46

### Degradation of Recyclable Carbon Fiber Composite Laminate

In a round-bottomed flask, a piece of recyclable carbon fiber composite laminate sample (1.0 g) from Example 37 was imerged in a mixture of 5 mL methanesulfonic acid and 90 mL hexanol. After heated at 135° C. for 4 hours, the degradation was almost complete and solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 0.95 g, giving a mass recovery yield of 95%. The surface of the recycled carbon fiber was very clean and essentially free of signs of damage.

### Example 47

### Degradation of Recyclable Carbon Fiber Composite Laminate

In an autoclave, a piece of recyclable carbon fiber composite laminate sample (0.6 g) from Example 38 was imerged in a mixture of 0.1 mL concentrated hydrochloric acid and 90 mL ethylene glycol. After heated at 350° C. for 0.5 hours, the degradation was almost complete and the solution was filtered to separate the carbon fibres, and the filtrate was neutralized with 2% aqueous sodium hydroxide solution. The resultant suspension was filtered again, and the collected solid was washed with water and dried. The total mass of recovered carbon fibre and degraded resin was 0.57 g, giving a mass recovery yield of 95%. The surface of the recycled carbon fiber was very clean and essentially free of signs of damage.

### Example 48

### Degradation of Recyclable Carbon Fiber Composite Laminate

In a round-bottomed flask, a piece of recyclable carbon fiber composite laminate sample (1.0 g) from Example 38 was imerged in a mixture of 30 mL concentrated hydrochloric acid and 70 mL ethylene glycol. After treated at room temperature for 120 hours, the degradation was almost complete and the solution was filtered to separate the carbon fibres, and the filtrate was neutralized with a 20% aqueous sodium hydroxide solution. The resultant suspension was filtered again, and the collected solid was washed with water and dried. The total mass of recovered carbon fibre and degraded resin was 0.95 g, giving a mass recovery yield of 95%.

### OTHER EMBODIMENTS

The invention has been described above with the reference to specific examples and embodiments, not to be constructed as limiting the scope of this invention in any way. It is understood that various modifications and additions can be made to the specific examples and embodiments disclosed without departing from the spirit of the invention, and such modifications and additions are contemplated as being part of the present invention.

1. An isocyanate compound having at least two isocyanate groups, wherein each of the isocyanate groups is bonded via a linker to a linear or cyclic oxy-carbohydro moiety that contains oxygen atoms not less than the isocyanate groups and optionally contains one or more heteroatoms each independently being S or N; each of the linker is alkylene, heteroalkylene, alkenylene, heteroalkyl, alkynylene, heteroalkynlene, arylene, or heteroaryl; the linear oxy-carbohydro moiety is alkyl, alkenyl, or alkynyl optionally substituted in the main chain with one or more heteroatoms, and is connected via oxo to each of the linkers that link the isocyanate groups to the oxy-carbohydro moiety; and the cyclic oxy-carbohydro is saturated, unsaturated, or aromatic ring or fused ring containing at least one oxygen atom in the ring and optionally contain an oxygen in a ring substituent that is bonded to the linker with a isocyanate group.

2. The isocyanate compound of claim 1, wherein the compound is of Formula (I): 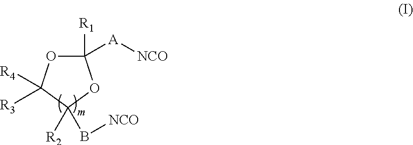
wherein,
m is 1, 2, 3, 4, or 5;
each of R₁, R₂, R₃ and R₄ independently is hydrogen, alkyl, cycloalkyl, heterocyclic, heterocyclic, alkenyl, cycloalkenyl, alkynyl, aryl, heteroaryl, alkylene-oxy-alkyl, alkylene-oxy-alkyl, alkylene-oxy-hetero-cyclic, alkylene-oxy-hetero-cycloalkyl, alkylene-oxy-alkenyl, alkylene-oxy-cycloalkenyl, alkylene-aryl, alkylene-oxy-heteroaryl, cycloalkylene-oxy-alkyl, cycloalkylene-oxy-cycloalkyl, cycloalkylene-oxy-heterocyclic, cycloalkylene-oxy-heterocycloalkyl, cycloalkylene-oxy-alkenyl, cycloalkylene-oxy-cycloalkenyl, cycloalkylene-oxy-aryl, cycloalkylene-oxy-heteroaryl, heterocycloalkylene-oxy-alkyl, heterocycloalkylene-oxy-cycloalkyl, heterocycloalkylene-oxy-heterocyclic, heterocycloalkylene-oxy-heterocycloalkyl, heterocycloalkylene-oxy-alkenyl, heterocycloalkylene-oxy-cycloalkenyl, heterocycloalkylene-oxy-aryl, heterocycloalkylene-oxy-heteroaryl, arylene-oxy-alkyl, arylene-oxy-cycloalkyl, arylene-oxy-heterocyclic, arylene-oxy-heterocycloalkyl, arylene-oxy-alkenyl, arylene-oxy-cycloalkenyl, arylene-oxy-aryl, or arylene-oxy-heteroaryl; or
R₃ and R₄, together with the carbon atom to which they are bonded, form a 3-7 membered ring optionally containing one or more heteroatoms each of which is independently S, O, or N; or
R₁ and A; together with the carbon atom to which they are bonded, form a 3-7 membered ring optionally containing one or more heteroatoms each of which is independently S, O, or N; or
R₂ and B, together with the carbon atom to which they are bonded, form a 3-7 membered ring optionally containing one or more heteroatoms each of which is independently S, O, or N; or
each of A and B independently is alkylene, alkylene-hetero-alkylene, alkenylene, alkenylene-hetero-alkenylene, alkylene-hetero-alkenylene, alkynylene, cycloalkylene, alkylene-cycloalkylene, alkylene-cycloalkylene-alkylene, alkenylene-cycloalkylene, alkenylene-cycloalkylene-alkenylen, alkylene-cycloalkylene-alkenylene, alkynylene-cycloalkylene, alkynylene-cycloalkylene-alkynylene, heterocycloalkylene, alkylene-heterocycloalkylene, alkylene-heterocycloalkylene-alkylene, alkenylene-heterocycloalkylene, alkenylene-heterocycloalkylene-alkenylene, alkylene-heterocycloalkylene-alkenylene, alkynylene-heterocycloalkylene, alkynylene-heterocycloalkylene-alkynylene, cycloalkenylene, alkylene-cycloalkenylene, alkylene-cycloalkenylene-alkylene, alkenylene-cycloalkenylene, alkenylene-cycloalkenylene-alkenylene, alkylene-cycloalkenylene-alkenylene, alkynylene-cycloalkenylene, alkynylene-cycloalkenylene-alkynylene, heterocycloalkenylene, alkylene-heterocycloalkenylene, alkylene-heterocycloalkenylene-alkylene, alkenylene-heterocycloalkenylene, alkenylene-heterocycloalkenylene-alkenylene, alkylene-heterocycloalkenylene-alkenylene, alkynylene-heterocycloalkenylene, alkynylene-heterocycloalkenylene-alkynylene, Arylene, alkylene-arylene, alkylene-arylene-alkylene, alkenylene-arylene, alkenylene-arylene-alkenylene, alkylene-arylene-alkenylene, alkynylene-arylene, alkynylene-arylene-alkynylene, Heteroarylene, alkylene-heteroarylene, alkylene-heteroarylene-alkylene, alkenylene-heteroarylene, alkenylene-heteroarylene-alkenylene, alkylene-heteroarylene-alkenylene, alkynylene-heteroarylene, alkynylene-heteroarylene-alkynylene, carbonyl, or thiocarbonyl.

3. The isocyanate of claim 2, wherein m is 1; each of R₁, R₂, R₃ and R₄ is independently hydrogen or alkyl; or each A and B independently is alkylene or alkenylene.

4. The isocyanate compound of claim 1, wherein the isocyanate compound is of Formula (II): 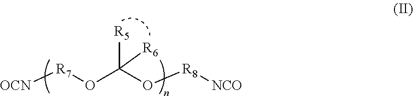
wherein:
each of R₅ and R₆ independently is hydrogen, alkyl, cycloalkyl, heterocyclic, heterocycloalkyl, alkenyl, cycloalkenyl, aryl, heteroaryl, alkyl-hetero-alkyl, alkynyl, alkylene, alkylene-hetero-alkylene, alkenylene, alkylene-hetero-alkenylene, alkynylene, or alkylene-hetero-alkynylene; or, R₅ and R₆, together with the carbon atom to which they are bonded, form a 3-7 membered ring optionally containing one or more heteroatoms each of which is independently S, O, or N;
n is 1, 2, 3, 4, 5, or 6;
each of R₇ and R₈ is independently alkylene, alkylene-hetero-alkylene, alkenylene, alkenylene-hetero-alkenylene, alkylene-hetero-alkenylene, alkynylene, cycloalkylene, alkylene-cycloalkylene, alkylene-cycloalkylene-alkylene, alkenylene-cycloalkylene, alkenylene-cycloalkylene-alkenylen, alkylene-cycloalkylene-alkenylene, alkynylene-cycloalkylene, alkynylene-cycloalkylene-alkynylene, heterocycloalkylene, alkylene-heterocycloalkylene, alkylene-heterocycloalkylene-alkylene, alkenylene-heterocycloalkylene, alkenylene-heterocycloalkylene-alkenylene, alkylene-heterocycloalkylene-alkenylene, alkynylene-heterocycloalkylene, alkynylene-heterocycloalkylene-alkynylene, cycloalkenylene, alkylene-cycloalkenylene, alkylene-cycloalkenylene-alkylene, alkenylene-cycloalkenylene, alkenylene-cycloalkenylene-alkenylene, alkylene-cycloalkenylene-alkenylene, alkynylene-cycloalkenylene, alkynylene-cycloalkenylene-alkynylene, heterocycloalkenylene, alkylene-heterocycloalkenylene, alkylene-heterocycloalkenylene-alkylene, alkenylene-heterocycloalkenylene, alkenylene-heterocycloalkenylene-alkenylene, alkylene-heterocycloalkenylene-alkenylene, alkynylene-heterocycloalkenylene, alkynylene-heterocycloalkenylene-alkynylene, arylene, alkylene-arylene, alkylene-arylene-alkylene, alkenylene-arylene, alkenylene-arylene-alkenylene, alkylene-arylene-alkenylene, alkynylene-arylene, alkynylene-arylene-alkynylene, Heteroarylene, alkylene-heteroarylene, alkylene-heteroarylene-alkylene, alkenylene-heteroarylene, alkenylene-heteroarylene-alkenylene, alkylene-heteroarylene-alkenylene, alkynylene-heteroarylene, alkynylene-heteroarylene-alkynylene, 1,4-alkyl substituted piperazine, carbonyl, or thiocarbonyl.

5. The isocyanate compound of claim 4, wherein each of R₅ and R₆ independently is hydrogen or alkyl; or, R₅ and R₆, together with the carbon atom to which they are bonded, form a 3-6 membered ring optionally containing one or more heteroatoms each of which is independently S, O, or N; n is 1; or each of R₇ and R₈ independently is alkylene, alkylene-hetero-alkylene, or alkenylene.

6. The isocyanate compound of claim 1, wherein the isocyante compound is of Formula (III):
(R₉)ₚR(—O—R₁₀—N═C═O)_{q} (III)
wherein:
R and the oxygen atoms (each between R and R₁₀) together constitute the linear or cyclic oxy-carbohydro moiety that optionally contains one or more heteroatoms each independently being S or N;
p is an integer no less than 0;
q is an integer of at least 3; when R is a carbon atom, the sum of p and q is 4;
each R₉ independently is hydrogen, alkyl, cycloalkyl, heterocyclic, heterocycloalkyl, alkenyl, cycloalkenyl, aryl, heteroaryl, alkyl-hetero-alkyl, alkynyl, alkylene, alkylene-hetero-alkylene, alkenylene, alkylene-hetero-alkenylene, alkynylene, or alkylene-hetero-alkynylene; or, two R₉, together with the carbon atom to which they are bonded, form a 3-7 membered ring optionally containing one or more heteroatoms each of which is independently S, O, or N; and
each R₁₀ independently is alkylene, alkylene-hetero-alkylene, alkenylene, alkenylene-hetero-alkenylene, alkylene-hetero-alkenylene, alkynylene, cycloalkylene, alkylene-cycloalkylene, alkylene-cycloalkylene-alkylene, alkenylene-cycloalkylene, alkenylene-cycloalkylene-alkenylen, alkylene-cycloalkylene-alkenylene, alkynylene-cycloalkylene, alkynylene-cycloalkylene-alkynylene, heterocycloalkylene, alkylene-heterocycloalkylene, alkylene-heterocycloalkylene-alkylene, alkenylene-heterocycloalkylene, alkenylene-heterocycloalkylene-alkenylene, alkylene-heterocycloalkylene-alkenylene, alkynylene-heterocycloalkylene, alkynylene-heterocycloalkylene-alkynylene, cycloalkenylene, alkylene-cycloalkenylene, alkylene-cycloalkenylene-alkylene, alkenylene-cycloalkenylene, alkenylene-cycloalkenylene-alkenylene, alkylene-cycloalkenylene-alkenylene, alkynylene-cycloalkenylene, alkynylene-cycloalkenylene-alkynylene, heterocycloalkenylene, alkylene-heterocycloalkenylene, alkylene-heterocycloalkenylene-alkylene, alkenylene-heterocycloalkenylene, alkenylene-heterocycloalkenylene-alkenylene, alkylene-heterocycloalkenylene-alkenylene, alkynylene-heterocycloalkenylene, alkynylene-heterocycloalkenylene-alkynylene, Arylene, alkylene-arylene, alkylene-arylene-alkylene, alkenylene-arylene, alkenylene-arylene-alkenylene, alkylene-arylene-alkenylene, alkynylene-arylene, alkynylene-arylene-alkynylene, Heteroarylene, alkylene-heteroarylene, alkylene-heteroarylene-alkylene, alkenylene-heteroarylene, alkenylene-heteroarylene-alkenylene, alkylene-heteroarylene-alkenylene, alkynylene-heteroarylene, alkynylene-heteroarylene-alkynylene, 1,4-alkyl substituted piperazine, carbonyl, or thiocarbonyl.

7. The isocyanate compound of claim 1, wherein the isocyanate is bis(4-isocyanatophenoxy)methane, bis(2-isocyanatoethoxy)methane, or 2,4-bis(isocyanatomethyl)-1,3-dioxolane, 1,1,1-tris(2-isocyanatoethoxy)ethane, 1,1,2-tris(2-isocyanatoethoxy)ethane, tetrakis(2-isocyanatoethoxy)methane, 1,1,1,2-tetrakis(2-isocyanatoethoxy)ethane, or 4,4′,4″-(ethane-1,1,1-triyltris(oxy))tris(isocyanatobenzene).

8. A method for preparing an isocyanate compound of claim 1, comprising the step of converting a compound of Formula (I-A) or Formula (II-A) or Formula (III-A) to the isocyanate compound, wherein R, R₁, R₂, R₃, R₄, A, B, R₅, R₆, R₇, R₈, R₉, R₁₀, m, n, p, and q, when present, are the same as those in the isocyanate compound.

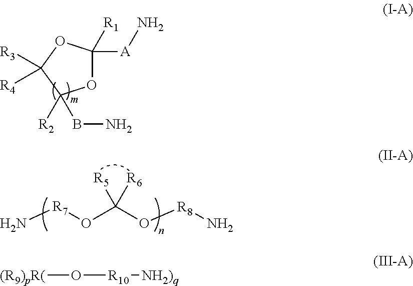

9. The method of claim 8, wherein the conversion of the compound of Formula (I-A) or Formula (II-A) or Formula (III-A) to the isocyanate compound is by reacting the compound of Formula (I-A) or Formula (II-A) or Formula (III-A) with phosgene, triphosgene, or trichloronethyl chloroformate, optionally at the presence of a catalyst.

10. The method of claim 9, wherein the molar ratio of the compound of Formula (I-A) or Formula (II-A) or Formula (III-A) to phosgene, triphosgene, or trichloronethyl chloroformate is 1:2˜100, the reaction temperature is in the range of −20˜150° C.; and the catalyst, when present, comprises an amine, a pyridine derivative, or N,N-dimethyl formamide.

11. A method for preparing an isocyanate compound of claim 1, comprising the step of converting a compound of Formula (I-B) or Formula (II-B) or Formula (III-B) to the isocyanate compound, wherein R, R₁, R₂, R₃, R₄, A, B, R₅, R₆, R₇, R₈, R₉, R₁₀, m, n, p, and q, when present, are the same as those in the isocyanate compound, and each X is independently hydroxyl, thiol, or trimethylsiloxy.

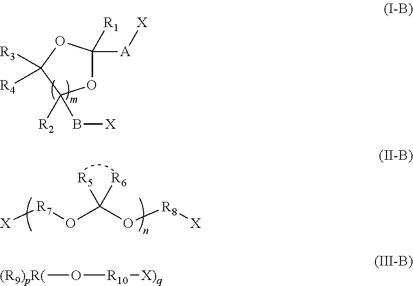

12. The method of claim 11, wherein the conversion of the compound of Formula (I-B) or Formula (II-B) or Formula (III-B) to the isocyanate compound is by reacting the compound of Formula (I-B) or Formula (II-B) or Formula (III-B) with tetrabutylammonium cyanate, optionally with the presence of a catalyst.

13. The method of claim 12, wherein the molar ratio of the compound of Formula (I-B) or Formula (II-B) or Formula (III-B) to tetrabutylammonium cyanate is 1:2˜100, the reaction temperature is in the range of −20˜150° C.; and the catalyst, when present, comprises a triazine compound.

14. A degradable polyurethane, wherein the polyurethane is made by polymerizing an isocyanate compound of claim 1 with a hydrogen-donating compound which comprises a dihydric alcohol, a polyhydric alcohol, polyetherpolyol, polyesterpolyol, binary mercaptan, polybasic mercaptan, phenol, carboxylic acid, urea, amide, diamine, or polyamine; and the polyurethane has a cleavable cross-linking structure of Formula (I-C) or Formula (II-C) or Formula (III-C), wherein R, R₁, R₂, R₃, R₄, A, B, R₅, R₆, R₇, R₈, R₉, R₁₀, m, n, p, and q, when present, are the same as those in the isocyanate compound.

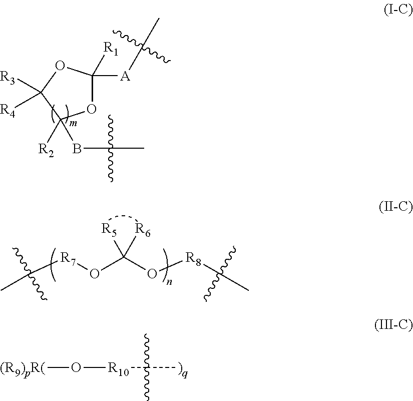

15. A degradable cross-linked polymer, wherein the polymer is made by polymerizing an isocyanate compound of claim 1 with an epoxy resin and a degradable curing agent; the epoxy resin comprises a glycidyl ether epoxy resin, a glycidyl ester epoxy resin, glycidyl epoxy amine epoxy resin, a trifunctional epoxy resin, a tetrafunctional epoxy resin, a novolac epoxy resin, an o-cresol formaldehyde epoxy resin, an aliphatic epoxy resin, an alicyclic epoxy resin, or a nitrogen-containing epoxy resin; and the degradable curing agent comprises an acetal or ketal aliphatic amine, an acetal or ketal aromatic amine or salt thereof, an acetal or ketal polyamine, a cyclic acetal or ketal amine, an acetal or ketal hydrazide, or hydrazone; and the cross-linked polymer has a cleavable cross-linking structure of Formula (I-C) or Formula (II-C) or Formula (III-C), wherein R, R₁, R₂, R₃, R₄, A, B, R₅, R₆, R₇, R₈, R₉, R₁₀, m, n, p, and q, when present, are the same as those in the isocyanate compound.

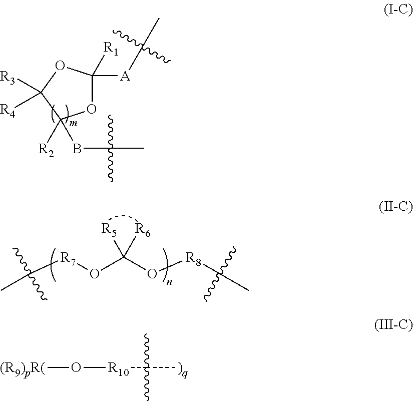

16. A method for degrading a polyurethane of claim 14, comprising the following steps:
(1) under the heating and stirring conditions, the degradable polyurethane is immersed in a mixed acid and solvent system for the degradation for 1˜600 hours at a temperature within the range of 15˜400° C., wherein the mass concentration of acid in the solvent 0.1˜99%;
(2) using an alkali solution to adjust the pH of the degradation solution to above 6 at a temperature within the range of 0˜200° C., wherein the mass concentration of alkali solution is 0.1˜99%.

17. The method of claim 16, wherein the acid comprises hydrochloric acid, hydrobromic acid, hydrofluoric acid, acetic acid, trifluoroacetic acid, lactic acid, formic acid, propionic acid, citric acid, methanesulfonic acid, p-toluenesulfonic acid, nitric acid, sulfuric acid, sulfurous acid, phosphoric acid, perchloric acid, benzoic acid, salicylic acid, or phthalic acid; the solvent system comprises methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, di methyl sulfoxide, tetrahydrofuran, methyl tetrahydrofuran, glycerol, or dioxane; the alkali comprises lithium hydroxide, sodium hydroxide, potassium hydroxide, calcium hydroxide, sodium carbonate, sodium bicarbonate, potassium carbonate, potassium bicarbonate, or ammonia; the solvent of the alkali solution comprises methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, di methyl sulfoxide, tetrahydrofuran, methyl tetrahydrofuran, glycerol, or dioxane.

18. The method of claim 16, wherein in step (1), the heating temperature is within the range of 80˜150° C., the heating time is within the range of 4˜8 hours, and the mass concentration of acid in the solvent 0.5˜20%; in step (2), the temperature is within the range of 5˜50° C., the final pH value after adjustment with the alkali solution is in the range of 6˜12, and the mass concentration of alkali solution is within the range of 5˜30%.

19. A recyclable reinforced composite material comprising a polyurethane of claim 14 or a cross-linked polymer of claim 15, a reinforcing material, and an auxiliary material, wherein the reinforcing material comprises carbon nanotubes, boron nitride nanotubes, carbon black, metal nano-particles, metal oxide nanoparticles, organic nanoparticles, iron oxide, glass fibers, carbon fibers, natural fibers, synthetic fibers and fabric made therefrom; and the auxiliary material comprises an accelerator, a diluent, a plasticizer, a toughening agent, a thickening agent, a coupling agent, a defoamer, a flatting agent, an ultraviolet absorber, an antioxidant, a brightener, a fluorescent agent, a pigment, or a filler.

20. A method for recycling a reinforced composite material of claim 19, comprising the steps of:
(1) under the heating and stirring conditions, immersing the reinforced composite material in a solution comprising an acid and a solvent and then heating the mixture at a temperature within the range of 15˜400° C. for 1˜600 hours to give rise to a degradation solution, wherein the mass concentration of acid in the solution is 0.1˜99%;
(2) using an alkali solution of 0˜200° C. to adjust the pH value of the degradation solution from step (1) to be greater than 6 to obtain a precipitate, wherein the mass concentration of the alkali in the alkali solution is 0.1˜99%;
(3) separate, wash and dry the precipitate obtained in step (2).

21. The method of claim 20, wherein the acid comprises hydrochloric acid, hydrobromic acid, hydrofluoric acid, acetic acid, trifluoroacetic acid, lactic acid, formic acid, propionic acid, citric acid, methanesulfonic acid, p-toluenesulfonic acid, nitric acid, sulfuric acid, sulfurous acid, phosphoric acid, perchloric acid, benzoic acid, salicylic acid, or phthalic acid; the solvent comprises at least one of methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, di methyl sulfoxide, tetrahydrofuran, methyl tetrahydrofuran, glycerol, or dioxane; the alkali comprises lithium hydroxide, sodium hydroxide, potassium hydroxide, calcium hydroxide, sodium carbonate, sodium bicarbonate, potassium carbonate, potassium bicarbonate, or ammonium hydroxide; and the alkali solvent comprises methanol, ethanol, ethylene glycol, propanol, isopropanol, butanol, isobutanol, t-butanol, pentanol, hexanol, heptanol, octanol, nonanol, benzyl alcohol, phenethyl alcohol, p-hydroxymethyl benzene, m-hydroxymethyl benzene, o-hydroxy benzene, p-hydroxyethyl benzene, m-hydroxyethyl benzene, o-hydroxyethyl benzene, water, N,N-dimethylformamide, N,N-dimethylacetamide, N-methylpyrrolidone, di methyl sulfoxide, tetrahydrofuran, methyl tetrahydrofuran, glycerol, or dioxane.

22. The method of claim 21, wherein in step (1), the mass concentration of acid in the solvent is within the range of 0.5˜20%, the temperature is within the range of 80˜200° C., and the reaction time is 2˜12 hours; and in step (2), the mass concentration of alkali solution is within the range of 5˜30%, the temperature is within the range of 5˜60° C.
